(19) United States
(12) Patent Application Publication
(10) Pub. No.: US 2025/0090230 A1
(43) Pub. Date: Mar. 20, 2025

(54) Lithotripsy Laser Spectroscopy in Situ

(71) Applicant: C. R. Bard, Inc., Franklin Lakes, NJ (US)

(72) Inventor: Paul R. Hess, Snellville, GA (US)

(21) Appl. No.: 18/726,648

(22) PCT Filed: Jan. 5, 2022
(86) PCT No.: PCT/US22/11323

§ 371 Date: Jul. 3, 2024

(51) Int. Cl. A61B 18/26; A61B 18/00

(52) U.S. Cl. CPC A61B 18/26; A61B 2018/00511

(57) ABSTRACT

Disclosed herein are medical systems, devices, and methods for performing a laser lithotripsy procedure on a patient. A medical system includes a medical device, having optical fibers extending therealong, and the elongate medical device is configured for advancement along a urinary tract of a patient. The system further includes a console including one or more processors and logic stored in memory that, when executed, causes operations of the system that include (i) projecting a coherent light away from the distal end onto a kidney stone within the patient, (ii) receiving a reaction light signal emanating from the kidney stone in response to the coherent light, and (iii) processing the reaction light signal to determine therefrom a material composition of the kidney stone. The system is also configured to project a coherent light onto the kidney stone to cause fragmentation of the kidney stone and the operational settings of the coherent light may be based on the determined composition of the kidney stone.

### BACKGROUND

Laser lithotripsy is an established method for removing a kidney stone from a patient. Different types of kidney stones are composed of different materials. The different materials may respond differently to laser lithotripsy or more specifically the operational settings of a lithotripsy laser. As such, it may be advantageous to the assess the composition of the kidney stone before or during the laser lithotripsy procedure so that the settings of the lithotripsy laser may be adjusted for optimal fragmentation or ablation of the kidney stone. Optimal settings during laser lithotripsy may be advantageous in reducing the time required to perform the laser lithotripsy procedure and minimizing the risk of injuring the patient such as damaging healthy tissue.

The systems, devices and methods disclosed herein provide kidney stone composition assessment in situ and thus may provide for some of the advantages described above.

### SUMMARY

Briefly summarized, embodiments disclosed herein are directed to medical systems, devices, and methods for performing a laser lithotripsy procedure on a patient. According to some embodiments, the medical system includes an elongate medical device, having one or more optical fibers extending between a proximal end and a distal end, where the elongate medical device is configured for advancement along a urinary tract of a patient. The system further includes a console coupled with the elongate medical device at the proximal end, where the console includes one or more processors and a non-transitory computer-readable medium having stored thereon logic that, when executed by the one or more processors, causes operations of the system that include (i) projecting a coherent light away from the distal end onto a kidney stone within the patient, (ii) receiving a reaction light signal emanating from the kidney stone in response to the coherent light, and (iii) processing the reaction light signal to determine therefrom a material composition of the kidney stone.

In some embodiments, the elongate medical device is configured for advancement along a working channel of a ureteroscope.

The projected coherent light may be configured to cause fragmentation of the kidney stone and the coherent light may be projected from a plurality of optical fibers. In some embodiments, the one or more of the optical fibers includes a fiber optic laser.

The coherent light and/or the reaction light signal may include a plurality of wavelengths. In some embodiments, the reaction light signal is defined at least partially by reflections of the coherent light.

In some embodiments, processing the reaction light signal includes defining a spectroscopy signature for the kidney stone based on the reaction light signal. The spectroscopy signature may include a plurality of wavelengths, where each wavelength has a corresponding intensity. Processing the reaction light signal may further include comparing the spectroscopy signature with one or more spectroscopy signatures stored in memory, where the spectroscopy signatures stored in memory represent different material compositions of kidney stones, and as a result of the comparison, determining the material composition of the kidney stone.

In some embodiments, the operations further include (i) receiving input information, where the input information includes an independent composition assessment of the kidney stone after removal from the patient, (ii) relating the input information to the spectroscopy signature, and (iii) combining the spectroscopy signature with at least at least one spectroscopy signature stored in memory to enhance an accuracy of the at least one spectroscopy signature stored in memory.

In some embodiments, the reaction light signal is received through one or more of the optical fibers.

In some embodiments, the coherent light projected from a first subset of optical fibers is configured to cause fragmentation of the kidney stone, and the coherent light projected from a second subset of optical fibers is configured to induce the reaction light signal.

In some embodiments, projecting the coherent light to cause fragmentation of the kidney stone is performed in accordance with operational settings, the operational settings including one or more of a pulse frequency, a pulse duration, or a wavelength of the coherent light. In further embodiments, at least one of the operational settings is defined at least partially based on the determined material composition of the kidney stone.

Further described herein is a method performed by a medical system where the method includes (i) transmitting a coherent light along a urinary tract of a patient, (ii) projecting the coherent light onto a kidney stone disposed within the patient, (iii) receiving a reaction light signal emanating from the kidney stone in response to the coherent light, and (iv) processing the reaction light signal to determine therefrom a material composition of the kidney stone.

In some embodiments of the method, the coherent light and/or the reaction light signal includes a plurality of wavelengths. The reaction light signal may be defined at least partially by reflections of the coherent light.

In some embodiments of the method, processing the reaction light includes defining a spectroscopy signature for the kidney stone based on the reaction light signal, where the spectroscopy signature includes a plurality of wavelengths, each wavelength having a corresponding intensity.

In some embodiments of the method, processing the reaction light signal includes (i) comparing the spectroscopy signature with one or more spectroscopy signatures stored in memory of the system, where the spectroscopy signatures stored in memory represent different material compositions of kidney stones, and (ii) as a result of the comparison, determining the material composition of the kidney stone.

In some embodiments, the method further includes (i) receiving input information, where the input information includes an independent composition assessment of the kidney stone after removal from the patient, (ii) relating the input information to the spectroscopy signature, and (iii) combining the spectroscopy signature with at least one spectroscopy signature stored in memory to enhance an accuracy of the at least one spectroscopy signature stored in memory.

In some embodiments of the method, projecting the coherent light includes projecting the coherent light in accordance with operational settings configured to cause fragmentation of the kidney stone, where the operational settings include one or more of a pulse frequency, a pulse duration, or a wavelength of the coherent light. At least one of the operational settings may be defined at least partially based on the determined material composition of the kidney stone.

In some embodiments of the method, the system includes an elongate medical device configured for advancement along the urinary tract and the medical device includes one or optical fibers. The medical device may also be configured for advancement along a working channel of a ureteroscope.

In some embodiments of the method, at least optical fiber includes a fiber optic laser and in further embodiments, the reaction light signal is received through one or more of the optical fibers.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 is an illustrative embodiment of a medical system including an elongate medical device having optical fibers extending therealong, in accordance with some embodiments;

FIG. 2 illustrates an end view of the medical device of FIG. 1, in accordance with some embodiments;

FIG. 3A illustrates the medical device of FIG. 1 disposed within a working channel of ureteroscope performing a laser lithotripsy procedure, in accordance with some embodiments;

FIG. 3B illustrates the medical device of FIG. 1 disposed within a working channel of ureteroscope performing a laser spectroscopy procedure, in accordance with some embodiments;

FIG. 4 is an exemplary illustration of a spectroscopy signature, in accordance with some embodiments; and

FIG. 5 is a flowchart of a method of operations conducted by the medical system of FIG. 1, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a stylet disclosed herein includes a portion of the stylet intended to be near a clinician when the stylet is used on a patient. Likewise, a “proximal length” of, for example, the stylet includes a length of the stylet intended to be near the clinician when the stylet is used on the patient. A “proximal end” of, for example, the stylet includes an end of the stylet intended to be near the clinician when the stylet is used on the patient. The proximal portion, the proximal end portion, or the proximal length of the stylet can include the proximal end of the stylet; however, the proximal portion, the proximal end portion, or the proximal length of the stylet need not include the proximal end of the stylet. That is, unless context suggests otherwise, the proximal portion, the proximal end portion, or the proximal length of the stylet is not a terminal portion or terminal length of the stylet.

With respect to “distal,” a “distal portion” or a “distal end portion” of, for example, a stylet disclosed herein includes a portion of the stylet intended to be near or in a patient when the stylet is used on the patient. Likewise, a “distal length” of, for example, the stylet includes a length of the stylet intended to be near or in the patient when the stylet is used on the patient. A “distal end” of, for example, the stylet includes an end of the stylet intended to be near or in the patient when the stylet is used on the patient. The distal portion, the distal end portion, or the distal length of the stylet can include the distal end of the stylet; however, the distal portion, the distal end portion, or the distal length of the stylet need not include the distal end of the stylet. That is, unless context suggests otherwise, the distal portion, the distal end portion, or the distal length of the stylet is not a terminal portion or terminal length of the stylet.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random-access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

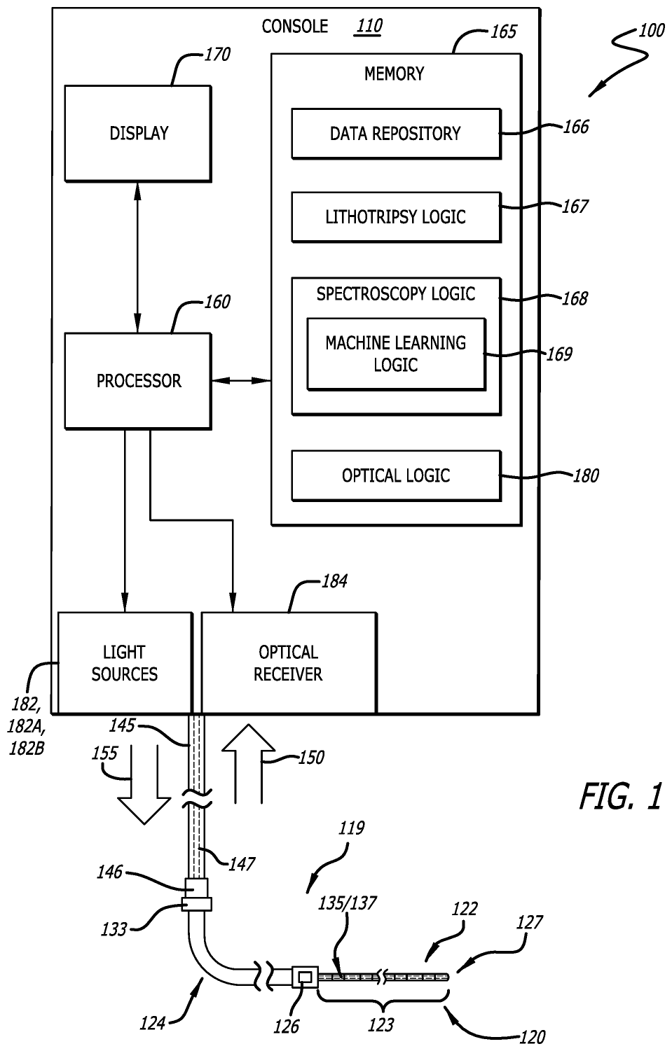

Referring to FIG. 1, an illustrative embodiment of a laser lithotripsy system (system) 100 including a laser spectroscopy functionality is shown in accordance with some embodiments. As shown, the system 100 generally includes a console 110 and a probe assembly 119 communicatively coupled to the console 110. The laser lithotripsy system 100 may be configured for utilization with an endoscope, such as a ureteroscope, for example. The system 100 may be employed for ablation or fragmentation of a kidney stone according to a lithotripsy mode of the operation. The system 100 may further be employed for determining a material composition of the kidney stone according to a spectroscopy mode of the operation.

An exemplary implementation of the console 110 includes a processor 160, a memory 165, a display 170 and optical logic 180, although it is appreciated that the console 110 can take one of a variety of forms and may include additional components (e.g., power supplies, ports, interfaces, etc.) that are not directed to aspects of the disclosure. An example of the console 110 is illustrated in U.S. Pat. No. 10,992,078, the entire contents of which are incorporated by reference herein. The processor or processors 160, with access to the memory 165 (e.g., non-volatile memory or non-transitory, computer-readable medium), is included to control functionality of the console 110 during operation. The display 170 may be a liquid crystal diode (LCD) display integrated into the console 110 and employed as a user interface to display information to the clinician, especially during a catheter placement procedure (e.g., cardiac catheterization). In another embodiment, the display 170 may be separate from the console 110. Although not shown, the console 110 may include a user interface that is configured to provide user control of the console 110. Such a user interface my be a graphical user interface integral to the display 170.

According to some embodiments, the content depicted by the display 170 may change according to which mode the probe 120 is configured to operate, i.e., the lithotripsy or spectroscopy more. In lithotripsy mode, the content rendered by the display 170 may constitute settings or operating conditions of the probe assembly 119. In the spectroscopy mode the content rendered by the display 170 may constitute kidney stone composition information.

The memory 165 includes a data repository 166, a lithotripsy logic 167, and a spectroscopy logic 168. In some embodiments, the spectroscopy logic 168 may further include machine learning logic 169. The lithotripsy logic 167 may define and control the operation of the system 100 for performing a lithotripsy operation of the system 100 and the spectroscopy logic 168 defines and controls the operation of the system 100 for determining a material composition of a kidney stone as further described below.

Optical logic 180 is configured to support the optical operability of the probe assembly 119. The optical logic 180 defines and controls the delivery of light to the probe assembly 119 and the receipt of optical signal information from the probe assembly 119.

As shown, both the light source(s) 182 and the optical receiver 184 are operably connected to the processor 160, which governs their operation. Also, the optical receiver 184 is operably coupled to provide the reaction light data to the data repository 166 for storage and processing by spectroscopy logic 168.

According to one embodiment, the probe assembly 119 includes an elongate probe (e.g., stylet) 120 extending along a distal portion 122 of the probe assembly 119. A proximal portion 124 of the probe assembly 119 includes a console connector 133 at a proximal end of the probe assembly 119. The console connector 133 optically couples with a connector 146 disposed at a distal end of interconnect 145 so that the probe assembly 119 is operably connected to the console 110 via a plurality of optical fibers 147 extending along the interconnect 145. The optical fibers 147 facilitate the transmission of light 155 from the light sources 182 and the return of the reflected light signals 150 to the console 110. The interconnect 145 may be flexible and relatively long (e.g., about two to ten feet in length) so that the console 110 may be located away from the patient for convenience.

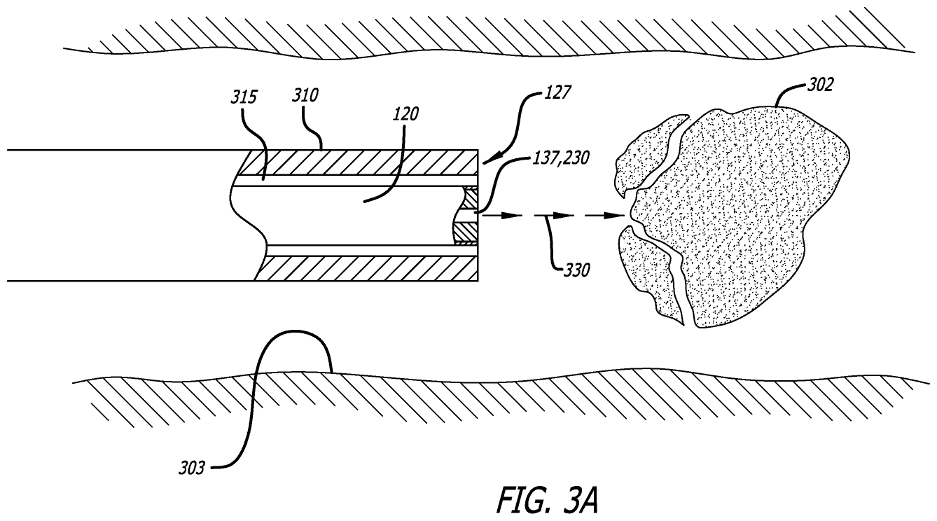

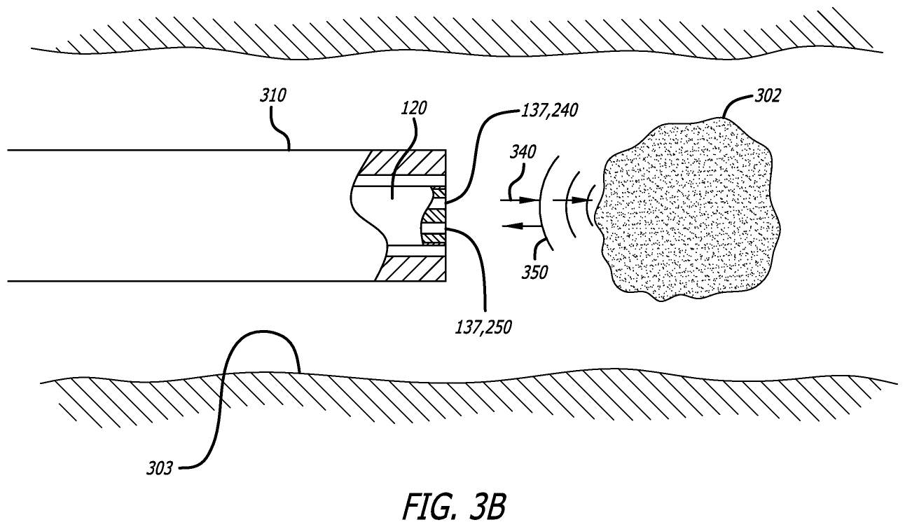

The stylet 120 is configured for insertion into a urinary tract of the patient body. As such, the stylet 120 defines a length 123 extending between an activation controller 126 at a proximal end 121 of the stylet 120 and a distal tip 127, and the length 123 is sufficient to extend from a location outside the patient to a location within a kidney of the patient. As stated above, the stylet 120 may be inserted into a working channel of a ureteroscope (see FIGS. 3A, 3B) and as such, the stylet 120 may be sized for insertion within and advancement along a working channel of the ureteroscope. More specifically, the length 123 may exceed the length of the ureteroscope, and a cross-sectional diameter of the stylet 120 may be sized for insertion into the working channel, i.e., less than a diameter of the working channel. In some embodiments, the cross-sectional diameter of the stylet 120 may be substantially less than an inside diameter of the working channel of the ureteroscope. The relatively small diameter of the stylet 120 with respect to the inside diameter of the working channel may provide for fluid flow through the working channel with the stylet 120 disposed therein.

According to one embodiment of the disclosure, an activation control 126, included on the probe assembly 119, may be used to define the operating mode of the system 100 and selectively alter operability of the display 170 by the clinician. For example, based on the mode of the system 100, the display 170 of the console 110 can be employed for laser lithotripsy or laser spectroscopy. In one embodiment, information from both modes may be displayed concurrently (e.g., at least partially overlapping in time).

In some embodiments, the stylet 120 may include a multi-core optical fiber core 135 extending along the length 123 of the stylet 120 to the distal tip 127. The multi-core optical fiber core 135 includes one or more core fibers 137. The core fibers 137 may include 1, 2, 3, 4, 5, or core fibers 137. In some embodiments, the core fibers 137 may include up to 10, 20, 30 or more core fibers 137.

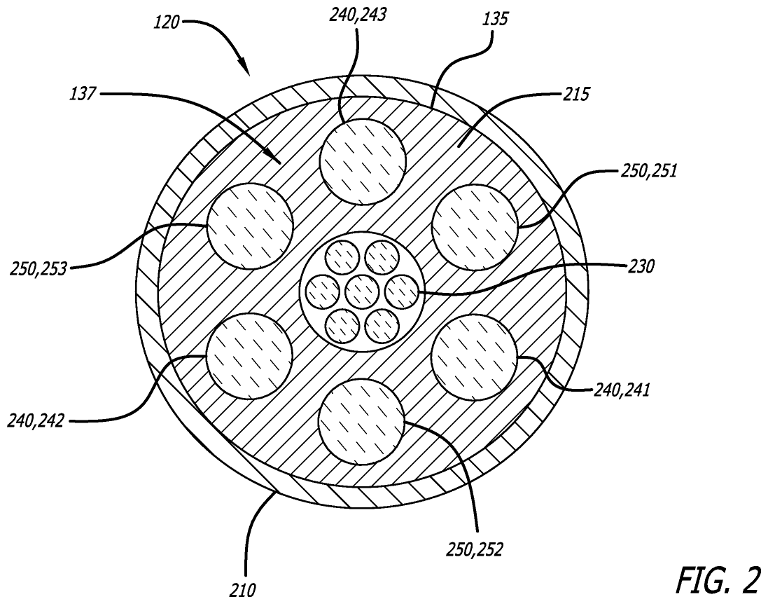

In the illustrated embodiment, the core fibers 137 may be divided up into subsets. For example, the core fibers 137 may include a first subset 230 (see FIG. 2) for performing laser lithotripsy, a second subset 240 facilitating the transmission of light 155 for performing laser spectroscopy, and a third subset 250 facilitating the return transmission of the reaction light signal(s) 150. In other embodiments, the core fibers 137 may not be divided into subsets, i.e., any or all of the core fibers 137 may be utilized in performing laser lithotripsy, transmitting light 155 for laser spectroscopy, and/or returning the reaction light signal 150.

The light source 182 may include separate light sources for the core fibers 137 of the first subset 230 and the core fibers 137 of the second subset 240. According to one embodiment, the light source 182 includes a light source 182A for providing light 155 to the first subset 230 and a second light source 182B for providing light 155 to the second subset 240.

In some embodiments, the first subset 230 may include one or more laser optical fibers. As such, light source 182A may provide light 155 in the form of non-coherent light to the subset 230 to stimulate radiation emission within the laser optical fibers. The one or more laser optical fibers of the subset 230 may receive light 155 individually or as one or more groups. In other embodiments, the subset 230 may not include laser optical fibers. In such embodiments, the light source 182A may provide a laser light (i.e., coherent light) to the core fibers 137 of the subset 230. As illustrated and described, the light source 182A and the core fibers 137 of the subset 230 may constitute a lithotripsy laser.

The second subset 240 may include core fibers suitable for transmitting coherent light along the stylet 120. As such, the light source 182B may include a coherent light source. The light source 182B may include any suitable type of coherent light source. For example, light source 182B may be a tunable swept laser, although other suitable light sources can also be employed in addition to a laser, including semi-coherent light sources, LED light sources, etc. In some embodiments, the light source 182B may include multiple coherent light sources that provide laser light at different wavelengths.

FIG. 2 illustrates a distal end view of the stylet 120 showing respective end views of the core fibers 137. As shown, the core fibers 137 may form a bundle of core fibers. In some embodiments, the core fibers 137 may define a specific arrangement. By way of example and not limitation, FIG. 2 illustrates one embodiment of an arrangement of the core fibers 137. In the exemplary embodiment, the subset 230 is centrally located within the cross section of the stylet 120 and subsets 240, 250 are distributed around the subset 230.

The subset 230 is illustrated as comprising a plurality of the core fibers 137. Although, as discussed above, the subset 230 may, in some embodiments, include a single core fiber. The subset 230 illustrates the core fibers as tightly bundled together. However, in other embodiments, the core fibers of the subset 230 may be dispersed among the core fibers of the subsets 240, 250.

In the illustrated embodiment, the subset 240 and the subset 250 each include three core fibers 137. However, as discussed above, the subset 240, 250 may each include one, two, four, or more core fibers 137 and the number of core fibers 137 of the subset 240 may be the same or different than the number of core fibers 137 of the subset 250. Similarly, while the core fibers 137 of the subsets 240, 250 are illustrated as evenly distributed in an alternating fashion, in other embodiments, the core fibers 137 of the subsets 240, 250 may be non-evenly distributed including randomly distributed.

In some embodiments, the stylet 120 may include a cladding 215 disposed between the core fibers 137 and the stylet 120 may also include a sheath 210 extending around the core fibers 137 and along the length 123 of the stylet 120.

FIG. 3A illustrates the stylet 120 during a laser lithotripsy process. As shown, in some embodiments, the stylet 120 may be employed with an endoscope such as the ureteroscope 310. In the illustrated embodiment, the stylet 120 is inserted within a urinary tract 303 extending toward a kidney stone 302 so that the distal tip 127 of the stylet 120 is located proximate the kidney stone 302 and is directed toward the kidney stone 302. In the illustrated implementation, the stylet 120 is disposed within a working channel 315 of the ureteroscope 310. In some implementations, the stylet 120 may be integral to the ureteroscope 310 so that the working channel 315 is available for other instruments, such as a fragment retrieval tool, for example. As shown, a laser light 330 projected from the core fibers 137 of the subset 230 impinges onto the kidney stone 302 to ablate, or fragment the kidney stone 302. In some embodiments, the laser light 330 may include a monochromatic beam having a wavelength between about 1900 nm (nanometers) and 2200 nm.

FIG. 3B illustrates the stylet 120 during a laser spectroscopy process. During the laser spectroscopy process laser light 340 is projected from the core fibers 137 of the subset 240 onto the kidney stone 302. The laser light 340 causes a reaction light signal 350 to emanate from the kidney stone 302 to be received by the core fibers 137 of the subset 250. The laser light 340 includes different wavelengths of light. The laser light 340 may include discrete wavelengths or a wavelengths extending across a continuous spectrum. In some embodiments, the laser light 340 may include a monochromatic beam having a wavelength between about 1900 nm and 2200 nm. In some embodiments, the laser light 340 may include the laser light 330. As such, the laser light 340 and the subset 240 of the core fibers 137 may be omitted.

The reaction light signal 350 may include any light (or any electromagnetic radiation) emanating from the kidney stone 302 in response to the laser light 340 impinging onto the kidney stone 302. For example, in some instances, the reaction light signal 350 may include reflections or scattering of the laser light 340, such as Raman scattering, for example. In some embodiments, the laser spectroscopy process may employ a Raman spectroscopy process. In other embodiments, the reaction light signal 350 may include emissions of light, such as fluorescence, for example. In still other instances, the reaction light signal 350 may be a combination of reflections and emissions of light. Similar to the laser light 340, the reaction light signal 350 may include multiple wavelengths including discrete wavelengths.

The spectroscopy logic 175 is configured to determine a material composition of the kidney stone 302. Kidney stones may include various materials. Most kidney stones are made of calcium compounds. Some kidney stones are made of uric acid. Other kidney stones are struvite stones occurring as a result of kidney or urinary tract infections. Still other kidney stones are composed of cystine. The composition of the kidney stone may be indicative of an optimal or preferred method of treatment or removal. For example, some kidney stones may be treated chemically while others may require mechanical treatment, such as laser lithotripsy, for example. Similarly, kidney stones of different compositions may be optimally with a different defined operational settings of the lithotripsy laser. As such, determining a composition of a kidney in situ may be advantageous in defining an optimal process for removal.

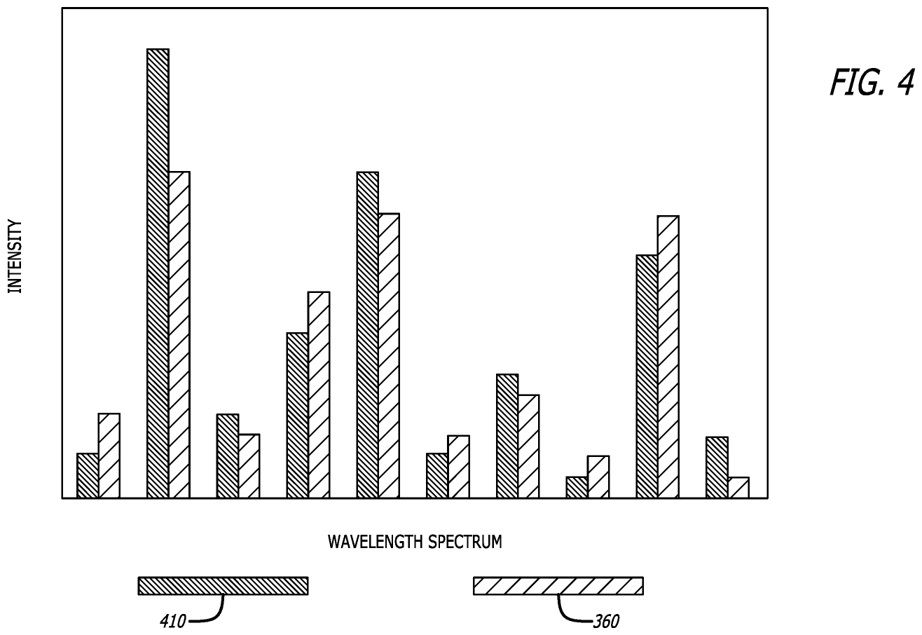

The spectroscopy logic 175 may define a spectroscopy signature (signature) 360 (see FIG. 4) based on the reaction light signal 350. The signature 360 may generally include intensities of light at different wavelengths. In other words, the reaction light signal 350 may include a first intensity at a first wavelength (or wavelength range), a second intensity at a second wavelength, and so on across a defined spectrum.

The data repository 166 may include multiple kidney stone signatures (i.e., spectroscopy signatures previously defined for various kidney stone types/compositions). The spectroscopy logic 175 may compare the signature 360 with the kidney stone signatures stored in the data repository 166 and a result of the comparison, determine that the signature 360 is consistent with (i.e., matches) one of the kidney stone signatures stored in the data repository 166. By way of example, spectroscopy logic 175 may compare the signature 360 with a calcium kidney stone signature stored in the data repository 166, and as a result of the comparison, the spectroscopy logic 175 may determine that the kidney stone 302 is a calcium kidney stone.

FIG. 4 is an exemplary bar chart illustrating a comparison of the signature 360 with a kidney stone signature 410 stored in the data repository. A wavelength spectrum extends along the x-axis and the y-axis indicates light intensity. As illustrated, each of the wavelengths includes an intensity. In some embodiments, the comparison may consist of comparing the intensities for each wavelength to if determine the intensity for each wavelength of the signature 360 matches (e.g., is within a defined tolerance of) the corresponding intensity for the kidney stone signature 410. If the corresponding intensities do match, then the spectroscopy logic 175 may determine that the material composition of the kidney stone 302 is consistent with a material composition of kidney stone type represented by the kidney stone signature 410.

In some embodiments, the spectroscopy logic 175 may include a machine learning logic 176. The machine learning logic 176 may record the signature 360 as a reference signature. Upon removal of the kidney stone, the type or composition of the kidney stone may be independently determined via a separate spectroscopy or chemical process. The results of the independent determination may be input into the system and related to the reference signature. Thereafter, the reference signature may be combined with a corresponding signature stored in memory to enhance an accuracy of the signature stored in memory. For example, the spectroscopy logic 175 may determine a kidney stone within the patient to be a calculus (i.e., a kidney stone formed of calcium) based on the comparison of the signature 360 with the exemplary signature 410. The clinician may obtain an independent determination after removal of the kidney stone that the kidney stone is indeed a calculus and input the independent determination into the system. The machine learning logic 176 may then combine the signature 360 (reference signature) as a training data set with the signature 410 to adjust the signature 410 (i.e., the intensity of each wavelength) to define a more accurate signature 410.

The lithotripsy logic 170 is configured to define the operation of the laser optical fibers of the subset 230 for performing laser lithotripsy on the kidney stone 302. Controlling the laser optical fibers 137 includes controlling the light source 182A. Controlling may include defining operational settings, such as a pulse frequency or a pulse duration of the lithotripsy laser, for example. Operational settings may also include utilizing any number of the laser optical fibers of the subset 230. Utilizing less than all of the laser optical fibers of the subset 230 may reduce the laser power applied to the kidney stone.

The composition of the kidney stone may also provide input for a laser lithotripsy treatment, i.e., the operational settings of the lithotripsy laser may be defined based on the determined composition of the kidney stone. In other words, the lithotripsy logic 170 may define the operational settings for the laser optical fibers of the subset 230 (lithotripsy laser) based on the signature 360.

Methods of the foregoing laser lithotripsy system include methods implemented in the laser lithotripsy system. For example, a method of the laser lithotripsy system includes a non-transitory CRM having the logic stored thereon that causes the laser lithotripsy system to perform a set of operations for determining a composition of a kidney stone via laser spectroscopy and performing laser lithotripsy via lithotripsy laser settings based on or partially on the determined composition of the kidney stone.

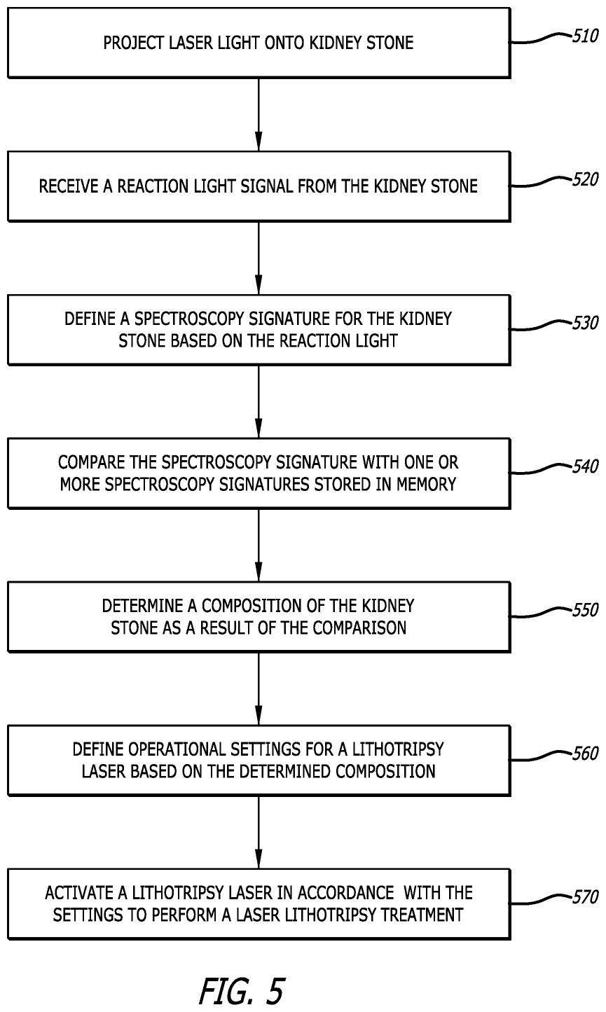

FIG. 5 illustrates a method 500 performed by the logic of the system (i.e., the lithotripsy logic 170, the spectroscopy logic 175, or a combination thereof) for ablating or causing fragmentation of a kidney stone. The system may project a light (e.g., a coherent light) along a urinary tract onto a kidney stone (or a fragment of a kidney stone) disposed with a patient (block 510). The system may project light having a defined wavelength or multiple defined wavelengths. Different wavelengths may be projected at the same time or different times. The light may be projected during a laser lithotripsy process or separate from the laser lithotripsy process.

The light may be transmitted along the stylet to a location proximate the kidney stone. In some embodiments, projecting the light may include activating one or more fiber optic lasers disposed along the stylet. In other embodiments, the light may originate from a laser within a console of the system and pass distally through one or more optical fibers disposed along the stylet.

The system may receive reaction light signal emanating from the kidney stone in response to the light projected onto the kidney stone (block 520). The reaction light signal may pass proximally through one or more optical fibers of the stylet to the console.

The logic may process the reaction light signal to define a spectroscopy signature for the kidney stone or fragment (block 530). In some embodiments, the spectroscopy signature is composed of a plurality of light wavelengths where each wavelength has a corresponding light intensity.

The logic may compare the spectroscopy signature for the kidney stone with one or more spectroscopy signatures stored in memory (block 540), where the spectroscopy signatures stored in memory represent different types of kidney stones or different material compositions of kidney stones. As a result of the comparison, the logic may determine that the kidney stone is of predetermined type or material composition (block 550).

The logic may define one or more operational settings for a laser lithotripsy light to be projected onto the kidney stone to ablate or cause fragmentation of the kidney stone (block 560). In some embodiments, the operational settings may include a wavelength of the laser light, a pulse rate or frequency, and/or a pulse duration. The operational settings may be defined based at least partially on the determined kidney stone type or material composition.

The logic may activate one or more laser optical fibers to project lithotripsy laser light onto the kidney stone in accordance with the defined settings to ablate or cause fragmentation of the kidney stone (block 570).

In some embodiments, the steps 510-570 may be performed multiple times to sufficiently ablate or break apart the kidney stone for removal.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A medical system, comprising:
an elongate medical device, comprising one or more optical fibers extending between a proximal end and a distal end, the elongate medical device configured for advancement along a urinary tract of a patient;
a console coupled with the elongate medical device at the proximal end, the console including one or more processors and a non-transitory computer-readable medium having stored thereon logic that, when executed by the one or more processors, causes operations including:
projecting a coherent light away from the distal end onto a kidney stone within the patient;
receiving a reaction light signal emanating from the kidney stone in response to the coherent light; and
processing the reaction light signal to determine therefrom a material composition of the kidney stone,
wherein the one or more of the optical fibers includes a fiber optic laser.

2. The system of claim 2, wherein the elongate medical device is configured for advancement along a working channel of a ureteroscope.

3. The system of claim 1, wherein the projected coherent light is configured to cause fragmentation of the kidney stone.

4. The system of claim 1, wherein the coherent light is projected from a plurality of optical fibers.

5. (canceled)

6. The system of claim 1, wherein the coherent light includes a plurality of wavelengths.

7. The system of claim 1, wherein the reaction light signal includes a plurality of wavelengths.

8. The system of claim 1, wherein the reaction light signal is defined at least partially by reflections of the coherent light.

9. The system of claim 1, wherein:
processing the reaction light signal includes defining a spectroscopy signature for the kidney stone based on the reaction light signal, and
the spectroscopy signature includes a plurality of wavelengths, each wavelength having a corresponding intensity.

10. The system of any of claim 9, wherein processing the reaction light signal includes:
comparing the spectroscopy signature with one or more spectroscopy signatures stored in memory, the spectroscopy signatures stored in memory representing different material compositions of kidney stones, and
as a result of the comparison, determining the material composition of the kidney stone.

11. The system of claim 9, wherein the operations further include:
receiving input information, the input information including an independent composition assessment of the kidney stone after removal from the patient;
relating the input information to the spectroscopy signature; and
combining the spectroscopy signature with at least at least one spectroscopy signature stored in memory to enhance an accuracy of the at least one spectroscopy signature stored in memory.

12. The system of claim 11, wherein the reaction light signal is received through one or more of the optical fibers.

13. The system of claim 1, wherein:
the coherent light projected from a first subset of optical fibers is configured to cause fragmentation of the kidney stone, and
the coherent light projected from a second subset of optical fibers is configured to induce the reaction light signal.

14. The system of claim 13, wherein projecting the coherent light to cause fragmentation of the kidney stone is performed in accordance with operational settings, the operational settings including one or more of a pulse frequency, a pulse duration, or a wavelength of the coherent light.

15. The system of claim 14, wherein at least one of the operational settings is defined at least partially based on the determined material composition of the kidney stone.

16. A method performed by a medical system, comprising:
transmitting a coherent light along a urinary tract of a patient;
projecting the coherent light onto a kidney stone disposed within the patient;
receiving a reaction light signal emanating from the kidney stone in response to the coherent light; and
processing the reaction light signal to determine therefrom a material composition of the kidney stone,
wherein the system includes an elongate medical device configured for advancement along the urinary tract, the medical device including one or optical fibers, and
wherein at least one of the one or more optical fibers includes a fiber optic laser.

17. The method of claim 16, wherein the coherent light includes a plurality of wavelengths.

18. The method of claim 16, wherein the reaction light signal includes a plurality of wavelengths.

19. The method of claim 16, wherein the reaction light signal is defined at least partially by reflections of the coherent light.

20. The method of claim 16, wherein:
processing the reaction light includes defining a spectroscopy signature for the kidney stone based on the reaction light signal, and
the spectroscopy signature includes a plurality of wavelengths, each wavelength having a corresponding intensity.

21. The method of claim 20, wherein processing the reaction light signal includes:
comparing the spectroscopy signature with one or more spectroscopy signatures stored in memory of the system, the spectroscopy signatures stored in memory representing different material compositions of kidney stones, and
as a result of the comparison, determining the material composition of the kidney stone.

22. The method of claim 20, further comprising:
receiving input information, the input information including an independent composition assessment of the kidney stone after removal from the patient;
relating the input information to the spectroscopy signature; and
combining the spectroscopy signature with at least at least one spectroscopy signature stored in memory to enhance an accuracy of the at least one spectroscopy signature stored in memory.

23. The method of claim 16, wherein projecting the coherent light includes projecting the coherent light in accordance with operational settings configured to cause fragmentation of the kidney stone, the operational settings including one or more of a pulse frequency, a pulse duration, or a wavelength of the coherent light.

24. The method of claim 23, wherein at least one of the operational settings is defined at least partially based on the determined material composition of the kidney stone.

25. (canceled)

26. The method of claim 16, wherein the medical device is configured for advancement along a working channel of a ureteroscope.

27. (canceled)

28. The method of claim 16, wherein the reaction light signal is received through one or more of the optical fibers.
